(19) United States
(12) Patent Application Publication
(10) Pub. No.: US 2026/0248474 A1
(43) Pub. Date: Aug. 27, 2026

(54) ALIGNERS FOR A 3D X-RAY IMAGING SYSTEM

(71) Applicant: 3DIO, Inc., Orem, UT (US)

(72) Inventors: Thomas L. YOUD, Holladay, UT (US); Casey Messick, Pleasant Grove, UT (US); Kolby Black, Salem, UT (US); Brad Allen, Orem, UT (US)

(21) Appl. No.: 19/062,647

(22) Filed: Feb. 25, 2025

(51) Int. Cl. A61B 6/58; A61B 6/00; A61B 6/51

(52) U.S. Cl. CPC A61B 6/587; A61B 6/4458; A61B 6/51; A61B 2560/0233

(57) ABSTRACT

This application describes dental X-ray devices and systems that use a rotating X-ray source to take two-dimensional (2D) images from multiple angles and then reconstruct a three-dimensional (3D) image using those 2D images. The X-ray imaging system contains an X-ray head with an X-ray source configured to rotate within the X-ray head, an X-ray detector and an aligner containing a proximal portion removably connected to the X-ray head, a distal portion configured to hold the X-ray detector, and an extender connecting the proximal and distal portions. The aligner is configured to rotate while attached to the front of the X-ray head, keeping the X-ray sensor aligned with the X-ray source as it rotates within the X-ray head and keeping the sensor at the same distance relative to the X-ray source when each X-ray image is captured by the sensor. Other embodiments are described.

### FIELD

This application relates generally to X-ray equipment, including X-ray devices and X-ray systems. More specifically, this application relates to an aligner for dental X-ray devices and systems that use a rotating X-ray source to take two-dimensional (2D) images from multiple angles and then reconstruct a three-dimensional (3D) image using those 2D images.

### BACKGROUND

X-ray imaging systems typically contain an X-ray source and an X-ray detector. X-rays (or other types of radiation used for imaging) are emitted from an X-ray tube in the X-ray source and impinge on the X-ray detector to provide an X-ray image of the object or objects that are placed between the X-ray source and the detector. The X-ray detector is often an image intensifier or even a flat panel digital detector.

Intra-oral radiography is a standard imaging technique in dentistry, with bite-wing and periapical X-rays considered the standard of care in dental practice. However, there are many features of the tooth anatomy that are not visible in standard intra-oral radiographs since they are only two-dimensional (2D) projections of a three-dimensional (3D) structure. Accordingly, 3D imaging is often used in some dental procedures. One form of 3D imaging, cone-beam computed tomography (CBCT), is becoming widely used in dental imaging. In CBCT, a patient's head is positioned between a large imaging detector and an opposing X-ray source. The detector and source rotate around the head while taking multiple 2D images. Using these 2D images, a 3D image of the patient's oral and maxillofacial anatomy can be reconstructed. This technique works very well for imaging the entire oral cavity and displaying the spatial relationships between the teeth and other bony structures located in the head of a patient. But it is often not used to image just a few (or a single tooth) because of the increased radiation that the patient is subjected to.

### SUMMARY

This application relates generally to X-ray equipment, including X-ray devices and X-ray systems. Specifically, this application describes an aligner for dental X-ray devices and systems that use a rotating X-ray source to take two-dimensional (2D) images from multiple angles and then reconstruct a three-dimensional (3D) image using those 2D images. The X-ray imaging system contains an X-ray head with an X-ray source configured to rotate within the X-ray head, an X-ray detector, and an aligner containing a proximal portion removably connected to the X-ray head, a distal portion configured to hold the X-ray detector, and an extender connecting the proximal and distal portions. The aligner is configured to rotate while attached to the front of the X-ray head, keeping both the X-ray sensor aligned with the X-ray source as it rotates within the X-ray head and keeping the sensor at the same distance relative to the X-ray source when each X-ray image is captured by the sensor.

### BRIEF DESCRIPTION OF THE DRAWINGS

The following description can be better understood in light of the Figures which show various embodiments and configurations of the X-ray tubes and X-ray devices in which they are used.

FIG. 1 shows a view of some embodiments of an X-ray imaging system used to create 3D images;

FIG. 2 shows some embodiments of an X-ray head in an X-ray imaging system;

FIGS. 3A-3B show other embodiments of an X-ray head in an X-ray imaging system;

FIGS. 4-5 show some embodiments of the housing and components contained in an X-ray head of an imaging system;

FIGS. 6A-6B show some embodiments of an aligner of an X-ray imaging system;

FIGS. 7A, 7B, and 8 show some embodiments of an X-ray imaging system mounted to equipment in a dental office;

FIGS. 9-10 show other embodiments of an aligner of a 3D dental imaging system;

FIGS. 11-12 shown the aligner in FIGS. 9-10 rotatably attached to the X-ray head of a 3D dental imaging system;

FIG. 13 shows one conventional 3D dental imaging system; and

FIG. 14 shows six degrees of freedom through which 3D dental imaging systems can rotate.

Together with the following description, the Figures demonstrate and explain the principles of the structures and methods described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices.

### DETAILED DESCRIPTION

The following description supplies specific details in order to provide a thorough understanding. Nevertheless, the skilled artisan will understand that the described X-ray systems can be implemented and used without employing these specific details. Indeed, the described systems and methods can be placed into practice by modifying the described systems and methods and can be used in conjunction with any other apparatus and techniques conventionally used in the industry. For example, while the description below focuses on X-ray devices and systems that can be used in imaging systems for dental imaging, they can be used for other purposes such as medical imaging, veterinary imaging, industrial inspection applications, and anywhere where X-ray radiography equipment is currently being used to generate a standard 2D X-ray image.

In addition, as the terms on, disposed on, attached to, connected to, or coupled to, etc. are used herein, one object (e.g., a material, element, structure, member, etc.) can be on, disposed on, attached to, connected to, or coupled to another object—regardless of whether the one object is directly on, attached, connected, or coupled to the other object or whether there are one or more intervening objects between the one object and the other object. Also, directions (e.g., on top of, below, above, top, bottom, side, up, down, under, over, upper, lower, lateral, orbital, horizontal, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

Dental X-ray radiography can be performed by positioning an X-ray source on one side of an object (e. g., a tooth or a set of teeth) and causing the X-ray source to emit X-rays through the teeth and toward an X-ray detector located on the other side of the teeth, either inside or outside of the mouth. As the X-rays pass through the teeth, jaws, and other tissues, they are absorbed to varying degrees depending on the composition of the material. The X-rays arriving at the X-ray detector form an image based on the cumulative absorption through the different materials and can provide a high level of detail of the tooth, bone, and supporting tissues. The images allow dentists to find cavities, examine tooth roots, evaluate the condition of the bony area around the tooth to determine if periodontal disease is present or a concern, and monitor the status of developing teeth, among other things.

Intraoral radiography (with the detector placed in the mouth of a patient) is the primary diagnostic method of dental imaging since it provides relatively high resolution and limited field of view images for most routine dental needs. Current 2D radiographs of objects (such as a tooth of a patient), however, are often ambiguous for detection of problems or defects because they are not usually able to visualize fractures in a tooth, and because they use planar projections, they can miss tooth curvature and other anomalies important to dental diagnosis and treatment. Despite many technological advances, the diagnostic accuracy of 2D dental X-ray imaging for the most common dental conditions has not improved in many years. Indeed, most 2D dental imaging often suffers from superposition of overlying and underlying structures due to the loss of spatial information in the depth or Z-axis dimension. This superposition of details is one major reason why 2D X-ray imaging has largely been replaced by 3D X-ray imaging technology for mammography and other medical applications.

Accordingly, 3D imaging that reconstructs a 3D volume from multiple 2D images has been developed to overcome many of the drawbacks of 2D imaging. Tomosynthesis is one type of X-ray imaging where the 3D images are reconstructed from 2D images of the patient taken from multiple perspectives within a scan angle typically ranging from about 10°to an upper limit of about 60°. Tomosynthesis improves the visibility of anatomical structures by reducing visual clutter from underlying and overlying normal anatomy. Some examples of current or developing clinical tomosynthesis applications include chest, abdominal, musculoskeletal, and female breast imaging or mammography. But tomosynthesis has not been widely adopted in dental imaging.

Cone-beam computed tomography (CBCT) is one 3D imaging process that has become widely used in dentistry. In CBCT, a patient's head is positioned between a large imaging detector and an opposing X-ray source. The detector and source rotate around the patient's head while taking multiple 2D images. Using these numerous 2D images, a 3D image of the patient's oral and maxillofacial anatomy can be reconstructed. This technique works very well for imaging the entire oral cavity and displaying the spatial relationships between the teeth and other bony structures located in the head of a patient. The CBCT technique is, therefore, often used for dental implants and orthodontic procedures where such spatial relationships are important. It is especially useful in surgical planning procedures such as dental implant, endodontic and orthodontic treatment planning, and evaluation of certain endodontic and pathological conditions.

There are, however, several disadvantages associated with CBCT relative to 2D radiography. These disadvantages include a much higher system cost, noise and artifacts from dental restorations/appliances (which reduce image quality), and significantly lower resolution relative to 2D images due to the placement of the sensor outside of the mouth and other factors. As well, CBCT procedures cannot be performed with the patient in the operatory chair and the patient has to be moved to where the CBCT equipment is located. Acquisition, reconstruction, and interpretation time for images from CBCT procedures are greatly increased relative to 2D imaging, thereby reducing clinical efficiency. And CBCT imaging also requires significantly higher radiation doses delivered to significantly more of the patient's tissues, significantly increasing the ionizing radiation burden for the patient. Additionally projecting X-rays through the entire head, as required in CBCT procedures, leads to high X-ray scattering and attenuation from non-resolving anatomy, further decreasing the spatial resolution in the CBCT image. The spatial resolution of CBCT is worse than intra-oral radiography using 2D radiographs with an intra-oral sensor positioned right next to the tooth of interest. For this reason, CBCT is not considered a diagnostic imaging modality for most dental pathologies. Thus, CBCT procedures are not often used to image a single tooth, or just a few teeth, due to the high radiation dose, the inconvenience and time involved, and the lower resolution image. So CBCT is not an effective and low-radiation-dose imaging tool for diagnosing dental pathologies and for planning dental procedures which only involve a single tooth or a few adjacent teeth.

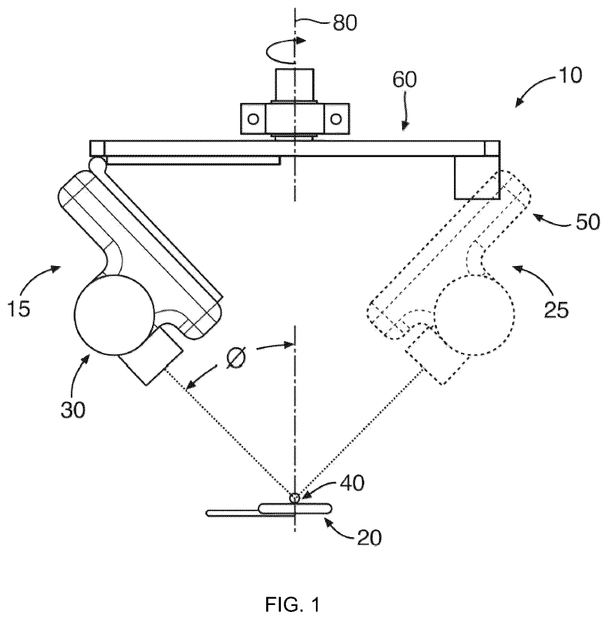

To overcome some of these limitations, the systems described herein provide a diagnostic imaging process that produces high-resolution 3D images using an aligner configured for a rotating X-ray source. Some embodiments of these imaging methods and associated devices or systems are illustrated in the figures. FIG. 1 shows some embodiments of an X-ray imaging system that can be used to carry out the imaging methods described herein. As illustrated in FIG. 1, the imaging system 10 contains an X-ray source at position 15 and position 25 around axis of rotation 80. In FIG. 1, the imaging system 10 comprises an imaging detector 20 that is located inside the mouth (not shown). The imaging detector 20 can be substantially stationary adjacent to the tooth (or teeth) 40 of a patient, or even completely stationary relative to the tooth using any stabilizing mechanism such as a bite block or other sensor holder device. The imaging system 10 also contains an X-ray source 30 that is located within a housing 50. The housing 50 can be connected to any type of support arm 60. In some configurations, the imaging system 10 can contain a removable power source (such as a battery) and optionally a power supply.

The imaging system 10 can contain any X-ray source 30 and X-ray detector 20 that allows the system 10 to take multiple 2D X-ray images or radiographs. The X-ray source 30 can contain any X-ray source that generates and emits X-rays, including a standard stationary anode X-ray source, micro-focus X-ray source, rotating anode X-ray source, and/or a carbon nano-tube or micro machined (Spindt cathode) X-ray source. In some embodiments, the X-ray source can operate with about 40 kV to about 90 kV and from about 1 mA to about 10 mA. In other embodiments, the X-ray source can operate with about 55 kV to about 75 kV and between about 3 mA and about 9 mA. In still other embodiments, the X-ray source can operate with about 60 kV to about 70 kV and between about 4 mA and about 7 mA. In some embodiments, the X-ray source and X-ray detector can be made modular so that different sizes and types of X-ray sources and X-ray detectors can be used.

The X-ray detector 20 can contain any detector that detects X-rays, including an image intensifier, CCD array, CMOS/scintillator array, a photon-counting detector, and/or a digital flat panel detector. In some configurations, the X-ray detector can have a substantially square shape with a length on one side ranging from about 2 cm to about 6 cm. In other configurations, though, the X-ray detector 20 does not need to have a substantially square shape but can have a substantially rectangular shape that provides an appropriate view of the patient's teeth. Typical dimensions for such a rectangular detector range from about 2 cm to about 3 cm on the short side and about 2.7 cm to about 4 cm on the long side.

In some configurations, the X-ray detector 20 can be synchronized with the X-ray source 30. Thus, the X-ray detector can be activated substantially at the same time that the X-ray source is activated, thereby capturing a 2D-projection image generated by the X-ray radiation passing through the patient's tooth/teeth and onto the detector.

In some configurations, the X-ray detector 20 can have quick readout speed. In the imaging systems described herein, this readout speed can range from about 5 to about 40 frames per second. This quick speed allows the necessary number of frames to be taken in a reasonable period of time. In other embodiments, the detector readout speed can be more than about 7 frames per second. In yet other embodiments, the detector readout speed can be more than about 12 frames per second. In still yet other embodiments, the detector readout speed can range from about 5 to about 20 frames per second. In even other embodiments, the readout speed can range between any combination or sub-combination of these amounts.

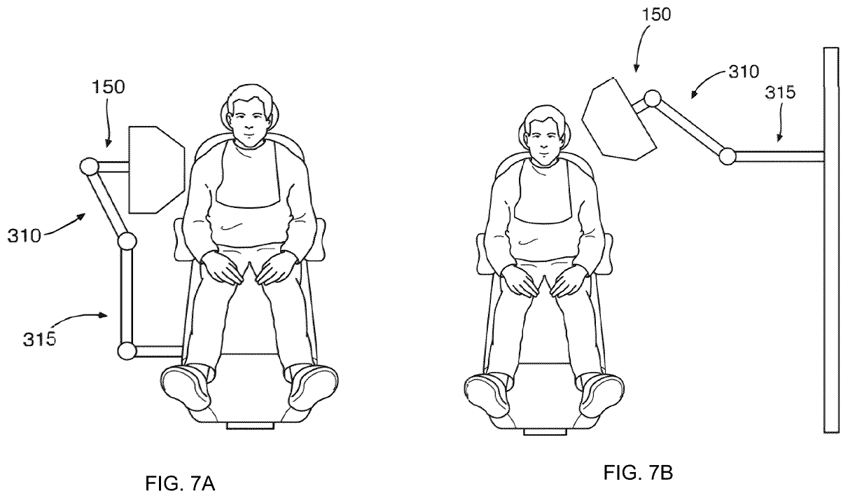

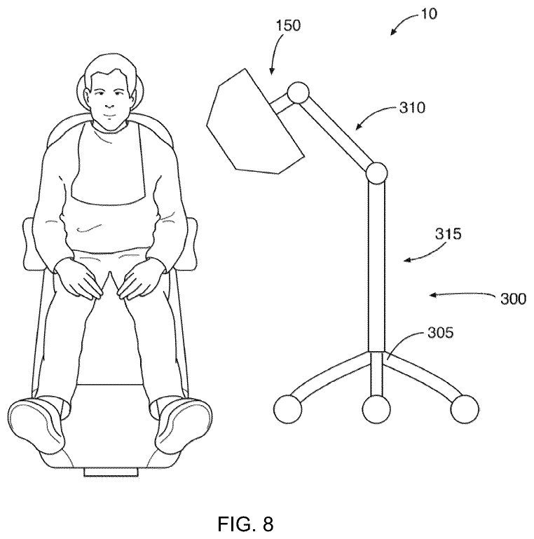

The support arm 60 can have any configuration that allows the X-ray source 30 in the housing to direct X-ray beams at the desired angle through the tooth (or teeth) and on the detector 20. In the embodiments shown in FIG. 1, the support arm 60 has a substantially straight configuration with the housing 50 connected to an end thereof. In other configurations, the support arm need not be straight and can have jointed or articulated sections, including those shown in FIGS. 7A-7B and 8. In yet other configurations, the housing 50 can be connected to the support arm 60 at any location other than the end.

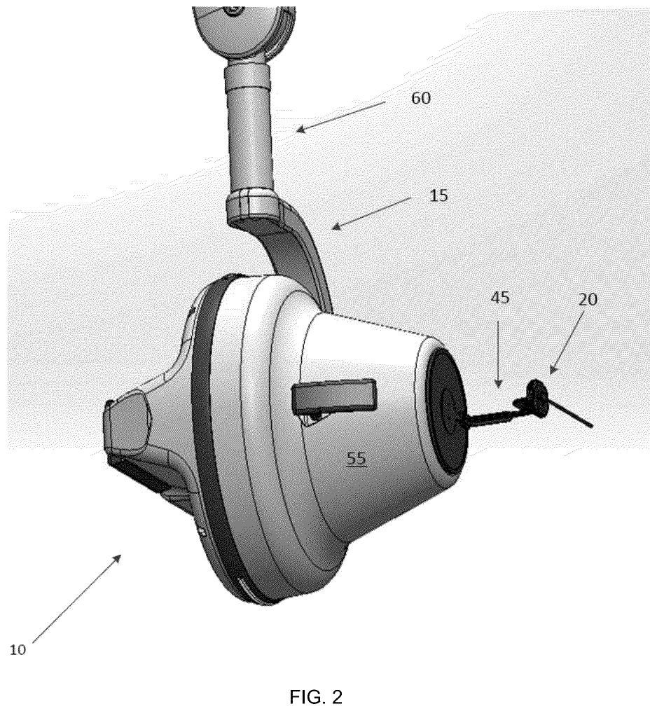

Some embodiments of an X-ray head 50 in the X-ray system 10 containing the X-ray source are shown in FIG. 2. In these embodiments, the support arm 60 of the X-ray imaging system 10 can have any configuration that allows the X-ray source 30 (not shown) in the X-ray head 50 to direct X-ray beams at the desired angle through the tooth (or teeth), soft tissue, and/or bone and on to the X-ray detector 20, while also aiding the operator to move the X-ray head 50 into any desired position to take images of a patient. In the embodiments shown in FIG. 2, the arm 60 has a bent configuration with the X-ray head 50 connected to an end thereof. In other configurations, the arm need not be bent and can have jointed or articulated sections as shown therein. In yet other configurations, the X-ray head 50 can be connected to the arm 60 at any location other than the end as shown in FIG. 2. In yet other configurations, the arm 60 can contain less or more sections that allows the arm 60 to move through a wider range of motions. The arm 60 can be configured with any length that allows it to be moved into any desired orientation and desired operational space.

In these embodiments, the X-ray imaging system 10 can further include a yoke 15 that is connected both to the arm 60 and the X-ray head 50, as shown in FIG. 2. The X-ray head 50 can be coupled to the arm 60 using the yoke 15. In these configurations, the X-ray head 50 can be configured to be removed from the yoke 15 so that the X-ray head 50 can then be removed from the remainder of the X-ray system 10. In these embodiments, the X-ray head 50 is connected to an aligner 45 that is connected to the X-ray detector 20. The aligner 45 helps keep the X-ray source in the X-ray head 50 aligned and properly positioned in both distance and angle with respect to the X-ray detector 20.

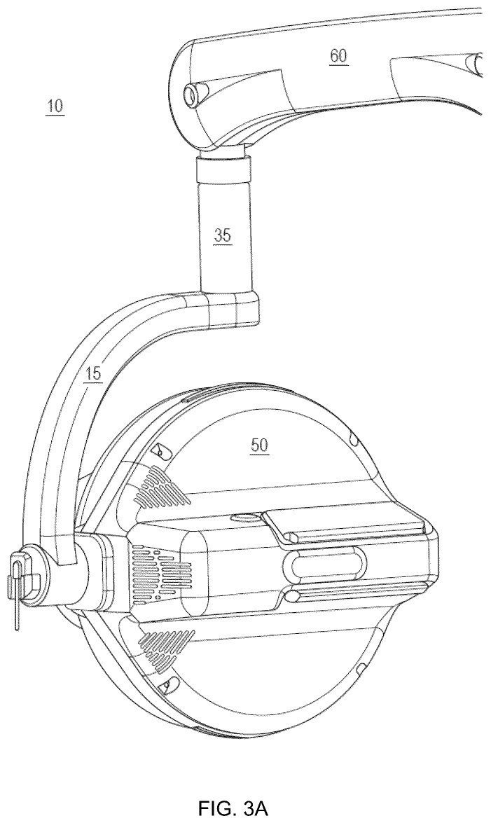

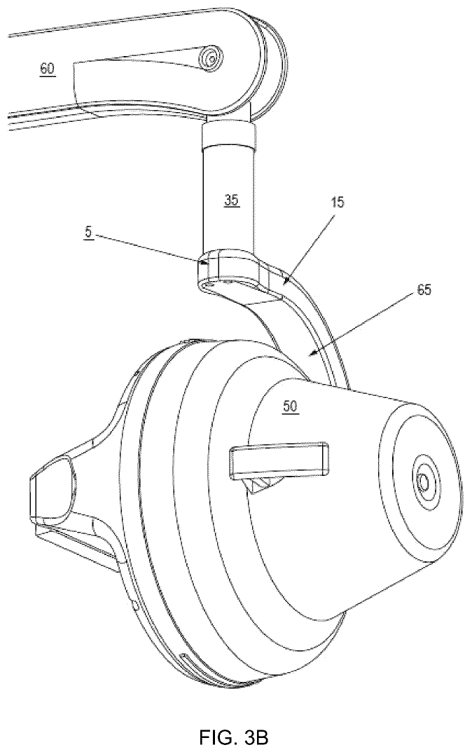

Other embodiments of an X-ray head containing the X-ray source are shown in FIGS. 3A-3B. In these embodiments, the support arm 60 of the X-ray imaging system 10 can have any configuration that allows the X-ray source 30 (not shown) in the X-ray head 50 to direct X-ray beams at the desired angle through the tooth (or teeth), soft tissue, and/or bone and on to the X-ray detector 20 (not shown), while also aiding the operator to move the X-ray head 50 into any desired position to take images of a patient. In the embodiments shown in FIGS. 3A-3B, the arm 60 has a bent configuration with the X-ray head 50 connected to an end thereof. In other configurations, the arm need not be bent and can have jointed or articulated sections as shown therein. In yet other configurations, the X-ray head 50 can be connected to the arm 60 at any location other than the end as shown in FIGS. 3A-3B. In yet other configurations, the arm 60 can contain less or more sections that allows the arm 60 to move through a wider range of motions. The arm 60 can also be configured with any length that allows it to be moved into any desired orientation and desired operational space.

In these embodiments, the X-ray imaging system 10 can further include a yoke 15 that is connected both to the arm 60 and the X-ray head 50, as shown in FIGS. 3A-3B. The X-ray head 50 can be coupled to the arm 60 using the yoke 15. In these configurations, the X-ray head 50 can be configured to be removed from the yoke 15 so that the X-ray head 50 can then be removed from the remainder of the X-ray system 10.

Some configurations of the yoke 15 and the arm 60 can be seen in FIGS. 3A-3B. In these configurations, a first end of the yoke 15 is connected to the X-ray head 50. A second end of the yoke 15 is connected to an arm extension 35 which is, in turn, connected to the arm 60. As explained herein, these configurations of the arm and yoke make it easy to disconnect the X-ray head from the rest of the X-ray system 10.

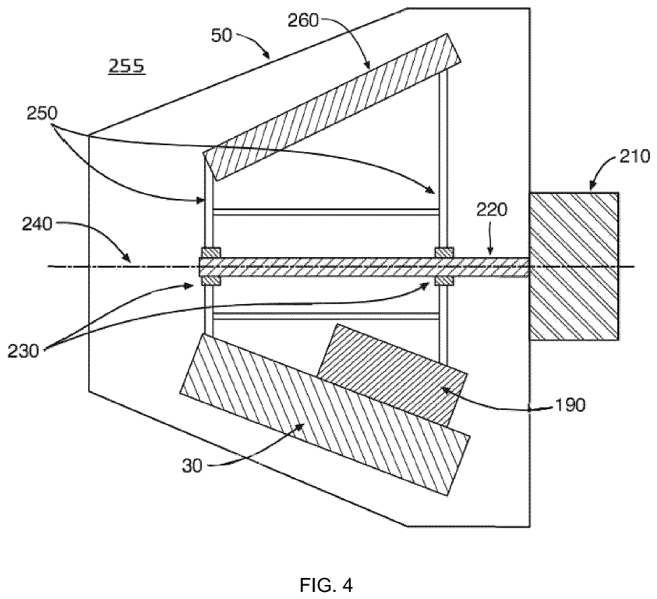

In the embodiments shown in FIG. 4, the X-ray source 30 can be contained in housing 50 of an X-ray head 255. The housing 50 can be configured with a first part enclosing the X-ray source 30 as shown in FIG. 4. The housing 50 also encloses a second part that contains a counterweight 260 for the X-ray source 30, power electronics 190, and other components, which facilitates smooth vibration-free rotary motion of the source 30. The X-ray source 30 and its associated power electronics 190 and the counterweight 260 are located as necessary on rotating mechanical assembly 250 which supports the X-ray source 30, the power electronics 190, counterweight 260, and other components (not shown) to properly balance the rotating mechanical assembly 250. The rotating mechanical assembly 250 is mounted to axle 220 (or other mechanical device to support the mechanical assembly) with an axis of rotation 240 using the bearings and/or electric motor assembly 230 to enable drive rotation of the mechanical assembly 250.

In the embodiments shown in FIG. 4, the housing 50 can be configured so that it is a single part that encloses both the X-ray source 30 and these components. In other configurations, the housing can be separated into different parts to contain the X-ray source 30 and other components. As shown in FIG. 4, the electronic components for control and power conditioning 210 can be located just outside of the housing 50. In other embodiments, these electronic components 210 can be located on the support arm or other convenient location. In yet other embodiments, these electronic components 210 can be located internal to the housing 50.

In some configurations, the power source and the power supply can be located on or in any supporting structure which the imaging systems might be used with. For example, the supporting electronics for the power source and the power supply, as well as the supporting electronics for the image display and for the wireless data upload described herein, can also be located internal or external to a support structure to which the housing 50 is connected, such as stand 300 shown in FIG. 8. Thus, in these configurations, the system 10 does not require an external power cord. Incorporating the power source (i.e., the battery), the power supply, and the supporting electronics all in or on the external structure allows the imaging systems to be portable and easily moved from one dental station to another. With such a configuration, the power source can easily be replaced or swapped such as by replacing a depleted battery with a fresh battery. Of course, if needed, the imaging system 10 can be configured so that it is alternately, or additionally, powered using external power from a power cord that is plugged into a wall outlet. In other configurations, multiple power supplies can be provided for the source, detector, and control electronics.

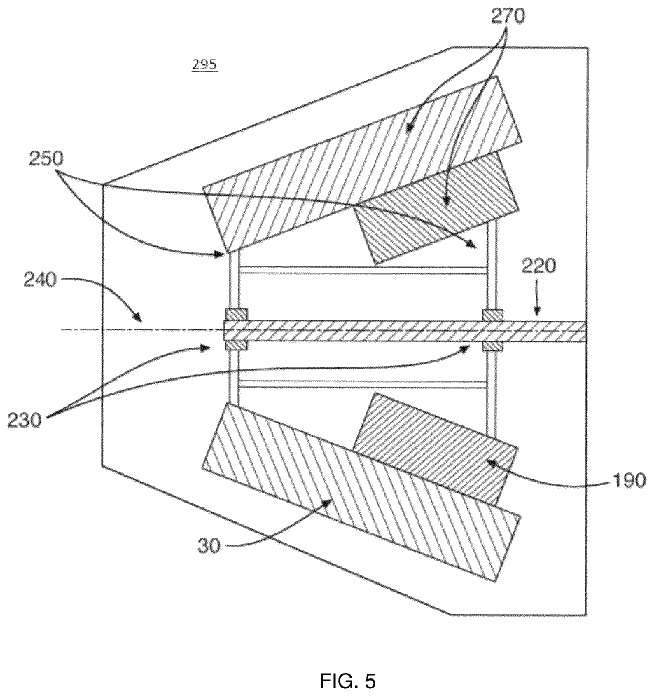

In some embodiments, multiple X-ray sources can be used in the 3D imaging systems. In these embodiments, as shown in FIG. 5, multiple X-ray sources (30, 270) enable a reduction in the mechanical rotation speed required to cover all of the desired X-ray source positions needed to generate the 3D image. These X-ray sources could be fired in an alternating manner or otherwise as required to obtain all of the desired 2D images from the various X-ray source locations within the head. The remainder of the components in FIG. 5 can be similar to those shown in FIG. 4, with the exception that the second X-ray source and its associated high voltage electronics 270 have replaced the counterweight 260.

The use of multiple X-ray sources 30 within the housing 50 can provide the benefit of reduced motion blur in the X-ray images obtained since the X-ray source is moving at a lower velocity than would be required with a single source. The use of multiple, substantially identical sources 30 would also negate the requirement for a counterweight since the multiple sources can be positioned to result in a balanced rotational system. More than two X-ray sources could be incorporated into the 3D imaging system, with the full 360 degrees of the circle being divided by the number of sources used so that the multiple sources are distributed evenly around the circular frame on which they are mounted. Of course, the use of multiple sources will increase the system overall cost and complexity, so the needs and constraints of the intended use will need to be considered in choosing the number of sources to be included within a particular 3D imaging system.

Indeed, as shown in the embodiments in FIG. 5, these electronic components 210 have been moved from the outside of the X-ray head 295 so that they are located internal to the housing of the X-ray head 295. The electronic components can be configured so that they rotate within the X-ray head 295 along with the X-ray source 30. In the embodiments shown in FIG. 5, these internal electronic components can include both a power supply and a power source (collectively labeled as 270). In these configurations, these internal electronic components can operate as a counterweight to the X-ray source 30, thus eliminating the separate counterweight that is shown in FIG. 4. In other configurations, the internal electronic components can be configured as a separate power source and power supply that, along with the X-ray source, all counterbalance each other. In yet other configurations, the X-ray source can be counter balanced by the power supply and the power source located near the motor or contained in the stand. In even other configurations, the power supply and the X-ray source could be combined and counterbalanced by the power source, or they could be separate (and counter balancing each other), with the power source either combined with the power supply or moved to an area outside of the rotation. In other words, there could be combinations of these three parts and any of them, except the X-ray source, could be placed outside of the rotation.

In some configurations, the imaging system can be removably or permanently mounted to any wall or any chair, as depicted in FIGS. 7A and 7B. The imaging system can be configured with any size, shape, and weight that will allow it to be mounted to a wall or chair, as shown in FIGS. 7A and 7B. The imaging system can also be configured with any size, shape, and weight so that it can be removably or permanently mounted to any desired support stand, as shown in FIG. 8. In one example, the imaging system can have a head volume of about 38 cm×about 38 cm×about 38 cm and a head weight of about 13 kg. In another example, the imaging system can have a head volume of about 20 cm×about 20 cm×about 18 cm and a head weight of about 5 kg. Of course, the head volume or head weight could have any amounts between these two examples.

Another example of an external support structure is illustrated in FIG. 8. In this figure, the imaging system 10 with a frame 150 can be connected to a stand 300. The stand 300 contains a base 305 and an arm 315 extending upwards towards an extension 310. The extension 310 is connected to the joint which is, in turn, connected to the frame 150 of the imaging system 10. In other configurations, the imaging system 10 can be connected to a movable support structure. In such configurations, the movable support structure can be configured to move across a floor while supporting the imaging system 10. Thus, the movable support structure can comprise one or more wheels, shelves, handles, monitors, computers, stabilizing members, limbs, legs, struts, cables, and/or weights (to prevent the weight of the imaging arm and/or any other component from tipping the movable support structure). Thus, the movable support structure could comprise a wheeled structure connected to a stand that contains the joint that is connected to the frame 150 of the imaging system 10.

The volume and weight of the imaging system should be minimized as much as possible for ease of use and ease of alignment by an operator. To keep the size and/or weight low, the imaging system can be equipped with small and light-weight components, including newer CMOS detectors that are much more sensitive, resulting in less dose to the patient than required with conventional CCD designs. Reducing the X-ray dose requirements also reduces the power demands on the X-ray source and other components, making it easier to reduce weight throughout the system.

The new CMOS detectors can also have very high readout speeds allowing for rapid collection and transmission of multiple 2D images. To achieve the imaging systems described herein, the actual X-ray emitter (or X-ray tube) should fit within a volume of about 13 cm×about 7 cm×about 8 cm and weigh less than about 1.9 kg. As well, CMOS detectors (or other technologies that are capable of fast image acquisition and transmission) with capability of 5 (or more) frames per second should be used. One way to achieve X-ray sources that meet these requirements would be to use a carbon-nanotube or Spindt-cathode (micro-machined silicon or similar technology) electron source within the X-ray source as this technology will simplify the X-ray tube design, enabling a smaller X-ray source and faster X-ray pulses.

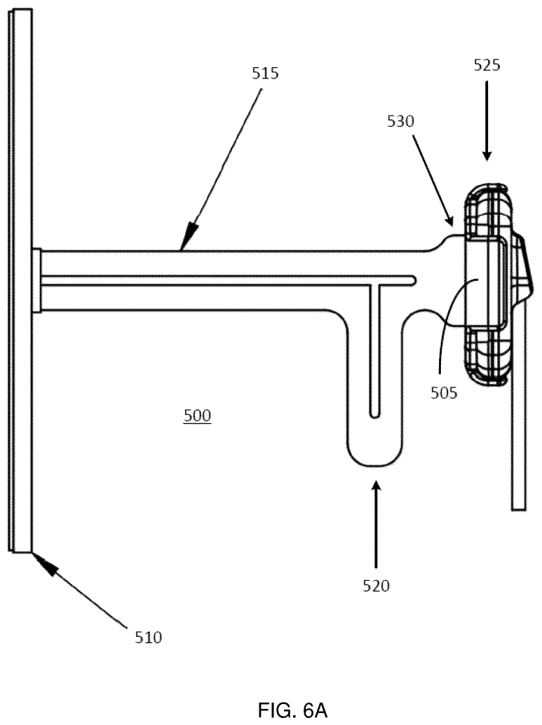

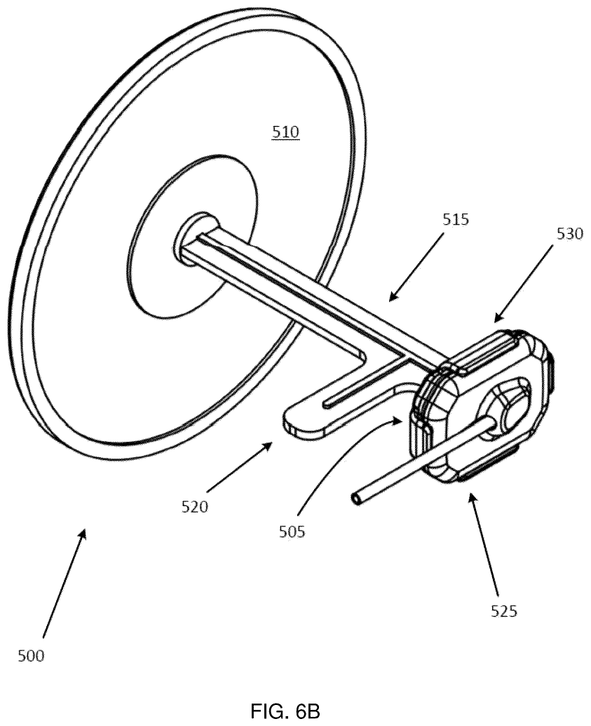

In some configurations, the X-ray imaging system can be configured so that the X-ray detector is attached or removably mounted to the X-ray head, as shown in FIGS. 6A-6B. In these configurations, the X-ray head connects with an aligner 500 that helps keep the X-ray sensor 505 substantially stationary and aligned behind the tooth of a patient while an X-ray image is taken. It also serves to create the proper positioning of the sensor relative to the X-ray source to meet the geometry requirements for the 3D reconstruction algorithm. The aligner 500 contains a first portion 510 that can be attached to the X-ray head. In some embodiments, the first portion can contain a ferrous metal (or other magnetic material) that clips the aligner 500 into the X-ray head. The aligner 500 also contains a second portion 515 (or extender) that is orientated substantially perpendicular to the first portion and extends towards the patient's mouth. The aligner 500 also contains a third portion 520 that is connected to the second portion 515 at one end and at the other end is connected to the sensor holder 525. This third portion 520 may also have extensions or other features intended for the patient to bite on or grip with the teeth to help hold the aligner in the proper position. The sensor holder 525 contains a housing, mounting feature, or clip that is configured to hold the X-ray sensor 505. In these figures, the extender 515 of the aligner 505 is configured to be connected to the middle of the first portion 510 of the aligner 500.

In some embodiments of the X-ray imaging systems, the X-ray source rotates within the X-ray head. In these embodiments, the aligner can be configured so that it assists with—and not detracts—from this rotation. The aligner can be removably attached to the front of the X-ray head to keep the sensor at the same distance relative to the X-ray source when each X-ray image is captured by the sensor. The aligner is also able to rotate while attached to the X-ray head so that the X-ray imaging system can accommodate varying patient head positions, different shots, and/or different X-ray views.

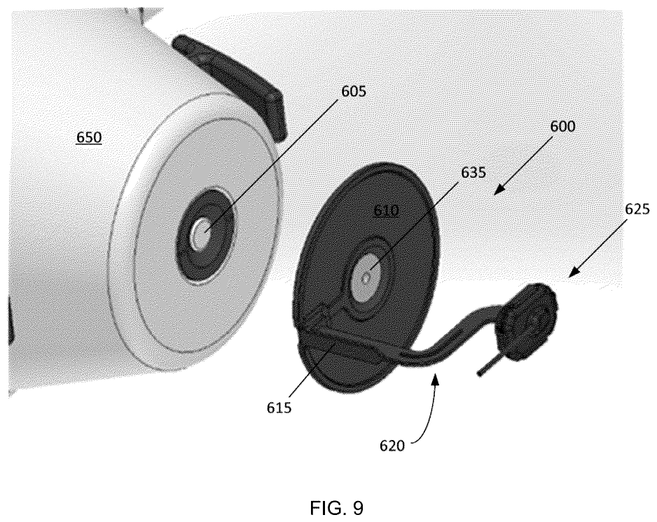

Some embodiments of these rotating aligners are shown in FIGS. 9-12. In these embodiments, the aligner 600 contain a first proximal portion (“first portion”) 610, a second portion 615, a third portion 620, and a distal portion with sensor holder 625. The first portion 610 of the aligner 600 is configured with a surface that substantially mates with the corresponding surface of the front end of the X-ray head 650. In the illustrated embodiments, the first portion 610 of the aligner 600 can also be configured with a shape that substantially matches the corresponding shape of the end of the X-ray head 650. In other embodiments, though, the first portion 610 can be configured with a shape that is smaller or bigger than the corresponding shape of the end of the X-ray head 650.

The first portion 610 of the aligner 600 can contain a center 635 that is configured to mate with the center 605 of the X-ray head 650. In these embodiments, the center 635 is sized and shaped to substantially match the center 605 of the X-ray head 650. In some configurations, the center 635 of the aligner can be made of any magnetic material that will magnetically attach the material of the aligner 600 to the material of the center 605 of the X-ray head 650. This magnetic material can be a magnetic grade of steel, such as 410, 420, or 440 steel, other metals with significant magnetic properties such as neodymium or nickel, and/or alloys thereof.

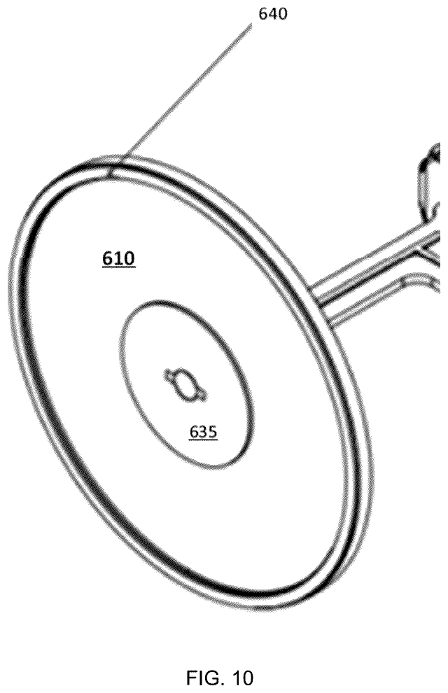

In other configurations, the aligner 600 can be magnetically attached to X-ray head 650 not using the center 605. In these configurations, the magnetic material can be moved from the center 635 to the outer part of the first portion so that it is a ring 640 as shown in FIG. 10. In yet other configurations, the ring of magnetic material can be located anywhere from the center 635 to the outer part of the first portion 610. In even other configurations, the metal ring does not have to be a continuous ring as shown in the figures but can be any combination of individual arc(s) of a circle. In still other configurations, the aligner 600 can be attached to the X-ray head 650 using any combination of these mechanisms.

By using these magnetic materials, the aligner can be removably attached to the X-ray head 650 so that the aligner 600 can partially or fully rotate while attached to the front of the X-ray head. This configuration allows for full or partial rotation of the aligner on the front end of the X-ray head 650 and helps keeping the sensor at the same distance, relative to the X-ray source, for each 2D image.

In some configurations, the aligner 600 can be attached to the X-ray head 650 with a magnetic connection using either one or multiple magnets central on the face of the head or multiple magnets distributed around a radius. The magnet(s) could be in the head of the X-ray system with magnetic metal in the aligner, or vice versa. In yet other configurations, the magnets could be located in both the head of the X-ray system and the aligner. The magnet can be combined with a mechanical guidance system to guide the user in attaching the aligner to the X-ray head. While the ring of magnets and the central magnet both allow for rotation, the outer ring may have a stronger hold because of the greater surface area of the magnetic material. In some configurations, the magnets could also be electromagnets, introducing the ability to change the strength of the attachment and the ability to turn them off completely to allow for a quick and easy connect/disconnect procedure.

Other methods of attaching or connecting the aligner 600 to the X-ray head 650 can be used in other embodiments. These other attachment methods could include mechanical elements such as a mechanical latch, a ball and spring locking into a detent, a vacuum or friction fit, or combinations thereof. These elements could even be combined with a magnetic attachment. The mechanical elements can provide a more consistent hold, but can be more difficult to use when a sterile barrier is used with the X-ray head.

As shown in the embodiments of FIG. 9, the first portion 610 of the aligner 600 can be configured with a disk shape that substantially matches the disk shape of the front of the X-ray head 650. The benefit of a disk shape includes added stability, as well as being able to easily notice any misalignment should the disk veer or lift off the edge of the X-ray head. In some configurations, the aligner can be molded from plastic so that it does not leave dark shadows in the X-ray images. In other configurations, it could be made from materials that will not affect the X-ray imaging process. In other embodiments, portions of the first portion could be removed to reduce weight and to avoid any X-ray interference while maintaining strength.

The aligner 600 also contains a second portion 615 (or extender) that is orientated substantially perpendicular to the first portion and extends towards the patient's mouth. In some embodiments, the second portion 615 may contain elements or features that help in sensor cable management.

The aligner 600 also contains a third portion 620 that is connected to the second portion 615 at one end, with the other end being connected to the distal portion with the sensor holder 625. This third portion 620 may also have extensions or other features intended for the patient to bite on or grip with the teeth to help hold the aligner 600 in the proper position. In other embodiments, the third portion 620 can have different configurations to allow better positioning at different areas in the mouth, allowing dentists to image specific teeth or anatomy in the mouth. The third portion 620 can also can have small hooks or other features used to hold the cable of the sensor so that the cable is not damaged by the patient biting and does not get in the way of the X-ray image.

The sensor holder 625 of the aligner 600 contains a housing, mounting feature, or clip that is configured to hold an X-ray sensor (or detector). The sensor can be attached to the aligner 600 through various methods such as a clip (or other mechanical connector) on some or all sides of the sensor, a clip in some or all corners of the sensor, a combination of clips on the sides and corners, and/or two clips on the side with stops to hold or prevent the sensor from sliding. In other embodiments, non-mechanical connector(s) could be used that do not detract from the X-ray imaging and alignment functions. For example, magnets could be used in the sensor and aligner so they connect in the desired manner.

In some embodiments, the sensor holder 625 can also contain and/or hold a calibration apparatus. The calibration apparatus (or fiducial marker) can be molded into the aligner, secured under a label, or held in place with epoxy or by other similar means. In some embodiments, the calibration apparatus has a calibration array with multiple calibration targets located within or on a substrate. Each calibration target can contain an element with an X-ray absorption different (either higher or lower) than that of the substrate. The multiple targets are in a fixed spatial relationship relative to each other. The calibration apparatus increases the accuracy of, and enables, the reconstruction of a three dimensional (3D) image from a series of two dimensional (2D) images taken by the X-ray imaging system. Additional details of this calibration apparatus are described in U.S. patent application Ser. No. 18/337,719, the entire disclosure of which is incorporated herein by reference.

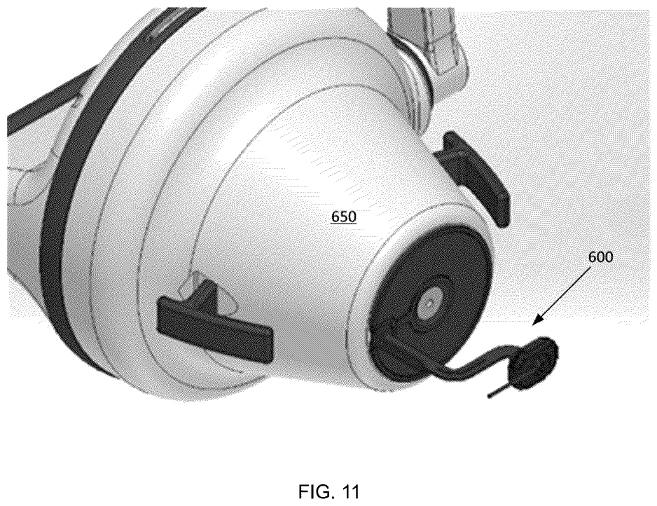

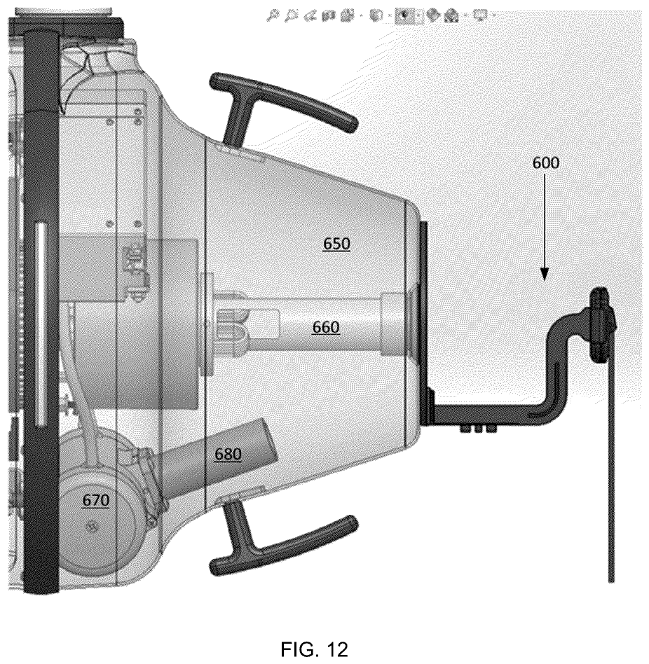

The position of the aligner 600 when connected to the X-ray head 650 is shown in FIGS. 11 and 12. As best seen in FIG. 12, the aligner 600 keeps the X-ray source 670 and the X-ray sensor in the sensor holder at a substantially constant distance relative to each other. The distance does not vary significantly because as the X-ray source 670 with collimator 680 rotates around the axis 660 of the X-ray head 650, the aligner 600 rotates along with it. The aligner 600 and the X-ray source 670 are kept in alignment with each other using the drive system internal to the X-ray head 650 that is described in U.S. patent application Ser. No. 18/977,278, filed on Dec. 11, 2024, the entire disclosure of which is incorporated herein by reference.

In some configurations, the aligner 600 for the X-ray imaging system can be configured so that it comprises a bite block. The bite block holds the imaging sensor in a patient's mouth when an X-ray image is taken. There are various designs of bite blocks that can be used that are similar to those used in 2D X-ray imaging systems. In 2D imaging systems, these designs can be used for bitewing (horizontal and vertical sensor alignment), anterior periapical, and posterior periapical images. Endodontists also employ a special aligner that is available with horizontal and vertical options for 2D X-ray imaging. Any of these examples could be used with appropriate modification to design an appropriate bite block for the aligner 600. In other configurations, the bite block can have a design that is unique for use in 3D imaging.

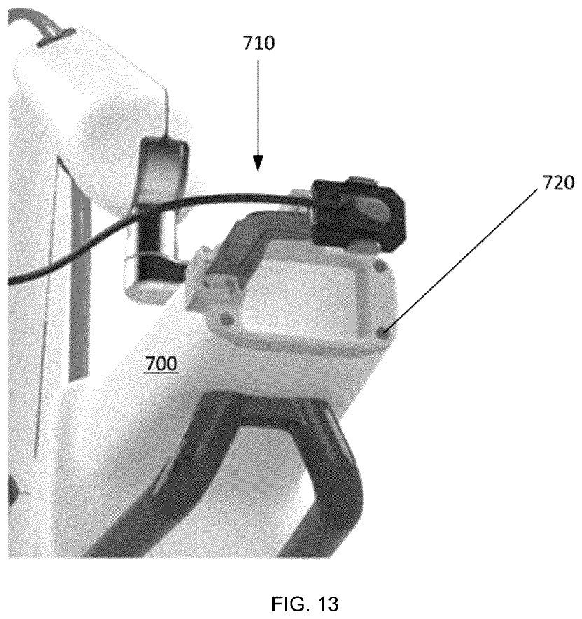

To capture X-rays in dental 2D imaging systems, dentists typically use an alignment aid to ensure the X-rays are directed at the sensor. This alignment aid often consists of a bite block and a ring connected together by a rod between them. The bite block is placed in the patient's mouth and then aligned with the X-ray tube which is placed concentrically within the ring of the aligner. A single X-ray image is then taken of the desired part of the patent's mouth. With 3D dental imaging, though, multiple 2D images need to be taken and then reconstructed into a 3D image. The sensor needs to be held in place in the patient's mouth through the whole process while taking the multiple images. In some conventional 3D dental imaging systems, the sensor is held in place using an alignment aid that is fixed to the front of the X-ray system by four magnets that are located in the corners. One example of these alignments aids is shown in FIG. 13, where the alignment aid 710 is held in place on the front of the X-ray head 700 using magnets 720 in each corner of the alignment aid on the front of the X-ray head 700.

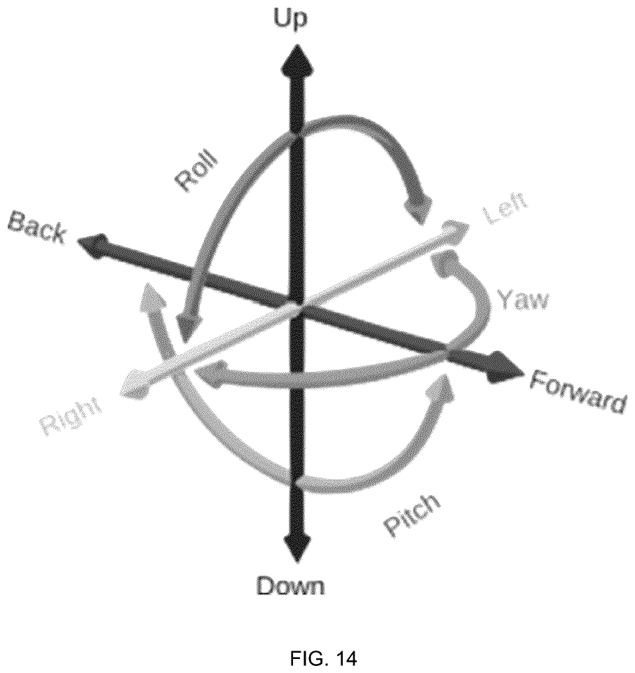

For the device in FIG. 13 to operate as a 3D dental imaging system, though, it has to move through all 6 degrees of freedom, as depicted in FIG. 14. The X, Y and Z movements are from the movement of the arm, the rotation about the yaw and pitch axis is through the yoke, and the roll axis using a gimble is at the connection of the head and yoke. In other words, to account for the movement of a patient's head, the X-ray head FIG. 13 device has to be constantly aligned with the sensor at the end of the aligner.

The 3D imaging systems described herein, though, do not need to move through all 6 degrees of freedom. Because the aligner is rotatably attached to the front of the X-ray head, it can rotate around or on the roll axis. Thus, the X-ray head of the 3D imaging systems described herein does not have to rotate along the roll axis and only has to move along the other 5 degrees of freedom. This allows these 3D imaging systems to place the sensor and aligner in the patient's mouth and then bring the X-ray system to the aligner and connect them with increased ease because that last degree of freedom does not need to be considered in the positioning process.

These 3D X-ray imaging systems with the aligners described herein exhibit several valuable features. With 3D intraoral X-ray technology being introduced into the market, there is a need to hold the sensor in correct alignment with the X-ray source. This alignment needs to be performed in a simple and a time efficient method similar to those currently employed in 2D X-ray methods. The aligner described herein provides this functionality since it rotates along the roll axis of the imaging system and is able to accommodate different patient head positions and different shots or X-ray views with relative ease.

Another helpful feature is that the 3D X-ray imaging systems are safer for patients because of the connection of the aligners described herein in. By ensuring the source and sensor are constantly aligned, the patient is not accidentally exposed to X-rays that are not usable in creating a clinically valuable image. The 3D X-ray imaging system can't initiate an X-ray sequence unless the aligner is properly connected to the front of the X-ray head. If this connection is broken during the middle of an X-ray capture, the X-ray source will stop irradiating, which prevents unnecessary exposure of the patient to X-rays.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. An aligner for a 3D X-ray imaging system, comprising:
a proximal portion configured to be removably connected to an X-ray head of an imaging system, the X-ray head containing an X-ray source configured to rotate within the X-ray head;
a distal portion configured to hold an X-ray sensor; and
an extender connecting the proximal and distal portions;
wherein the aligner is configured to rotate while attached to the X-ray head.

2. The aligner of claim 1, wherein the aligner is configured to be rotatably connected to the front of the X-ray head to keep the X-ray sensor aligned with the X-ray source as it rotates within the X-ray head.

3. The aligner of claim 1, wherein the aligner is configured to be rotatably connected to the front of the X-ray head to keep the sensor at the same distance relative to the X-ray source when each X-ray image is captured by the sensor.

4. The aligner of claim 1, wherein the proximal portion contains a center part made of a magnetic material configured to magnetically connect the aligner to the X-ray head.

5. The aligner of claim 1, wherein the proximal portion contains a circular ring made of a magnetic material configured to magnetically connect the aligner to the X-ray head.

6. The aligner of claim 5, wherein the circular ring is a continuous ring or one or more arcs of a circle.

7. The aligner of claim 1, wherein the proximal portion is removably connected to the X-ray head using a mechanical connector.

8. The aligner of claim 1, wherein the proximal portion is configured with a disk shape that substantially matches the shape of the front of the X-ray head.

9. The aligner of claim 1, wherein the distal portion is configured to contain or hold a calibration apparatus.

10. An X-ray imaging system, comprising:
an X-ray head containing an X-ray source configured to rotate within the X-ray head;
an X-ray detector;
an aligner containing a proximal portion removably connected to the X-ray head, a distal portion configured to hold the X-ray detector, and an extender connecting the proximal and distal portions, wherein the aligner is configured to rotate while attached to the X-ray head.

11. The X-ray imaging system of claim 10, wherein the aligner is configured to be rotatably connected to the front of the X-ray head to keep the X-ray sensor aligned with the X-ray source as it rotates within the X-ray head.

12. The X-ray imaging system of claim 10, wherein the aligner is configured to be rotatably connected to the front of the X-ray head to keep the sensor at the same distance relative to the X-ray source when each X-ray image is captured by the sensor.

13. The X-ray imaging system of claim 10, wherein the proximal portion contains a center part made of a magnetic material configured to magnetically connect the aligner to the X-ray head.

14. The X-ray imaging system of claim 10, wherein the proximal portion contains an outer circular ring made of a magnetic material configured to magnetically connect the aligner to the X-ray head.

15. The X-ray imaging system of claim 14, wherein the circular ring is a continuous ring or one or more arcs of a circle.

16. The X-ray imaging system of claim 10, wherein the proximal portion is removably connected to the X-ray head using a mechanical connector.

17. The X-ray imaging system of claim 10, wherein the proximal portion is configured with a disk shape that substantially matches the shape of the front of the X-ray head.

18. The X-ray imaging system of claim 10, wherein the distal portion is configured to contain or hold a calibration apparatus.
